SEQUENCE LISTING (17 sequences)

SEQ ID NO: 1 - DNA, 25 bp, Artificial
  aptamer
  misc_feature (1)..(1): n is T or EdU
  misc_feature (2)..(12): n is a sequence of 9 or 11 contiguous nucleotides comprising at least 5 guanidine nucleotides
  misc_feature (14)..(14): n is A or C
  misc_feature (17)..(17): n is T or EdU
  misc_feature (19)..(19): n is A or a deletion
  misc_feature (20)..(20): n is C or G
  misc_feature (21)..(21): n is T or EdU
  misc_feature (22)..(22): n is C or a deletion
  misc_feature (23)..(23): n is C or G
  misc_feature (24)..(24): n is G or T or EdU
  misc_feature (25)..(25): n is T or EdU
nnnnnnnnnnnngnccnannnnnnn

SEQ ID NO: 2 - DNA, 23 bp, artificial
  aptamer
  misc_feature (1)..(1): n is T or EdU
  misc_feature (15)..(15): n is T or EdU
  misc_feature (19)..(19): n is T or EdU
  misc_feature (23)..(23): n is T or EdU
ngggcaaagggcccnaagnccgn

SEQ ID NO: 3 - DNA, 23 bp, artificial
  aptamer
  misc_feature (1)..(1): n is T or EdU
  misc_feature (17)..(17): n is T or EdU
  misc_feature (20)..(20): n is T or EdU
  misc_feature (22)..(23): n is T or EdU
ncacgggcgggcgaccnacngnn

SEQ ID NO: 4 - DNA, 84 bp, Artificial
  aptamer
  misc_feature (31)..(31): n is T or EdU
  misc_feature (45)..(45): n is T or EdU
  misc_feature (49)..(49): n is T or EdU
  misc_feature (53)..(53): n is T or EdU
cacgacgcaagggaccacagggagggagggngggcaaagggcccnaagnccgnaacaaaaacacagcacgacaccgcagaggca

SEQ ID NO: 5 - DNA, 84 bp, Artificial
  aptamer
  misc_feature (28)..(28): n is T or EdU
  misc_feature (44)..(44): n is EdU
  misc_feature (47)..(47): n is EdU
  misc_feature (49)..(50): n is EdU
cacgacgcaagggaccacaggagagacncacgggcgggcgaccnacngnncagcccagaccgacagcacgacaccgcagaggca

SEQ ID NO: 6 - DNA, 84 bp, Artificial
  aptamer
cacgacgcaagggaccacagggagggagggtgggcaaagggccctaagtccgtaacaaaaacacagcacgacaccgcagaggca

SEQ ID NO: 7 - DNA, 84 bp, Artificial
  aptamer
  misc_feature (44)..(44): n is EdU
  misc_feature (47)..(47): n is EdU
  misc_feature (49)..(50): n is EdU
cacgacgcaagggaccacaggagagactcacgggcgggcgaccnacngnncagcccagaccgacagcacgacaccgcagaggca

SEQ ID NO: 8 - DNA, 38 bp, Artificial
  aptamer
  misc_feature (6)..(6): n is T or EdU
  misc_feature (20)..(20): n is T or EdU
  misc_feature (24)..(24): n is T or EdU
  misc_feature (28)..(28): n is T or EdU
gagggngggcaaagggcccnaagnccgnaacaaaaaca

SEQ ID NO: 9 - DNA, 42 bp, Artificial
  aptamer
  misc_feature (7)..(7): n is T or EdU
  misc_feature (23)..(23): n is EdU
  misc_feature (26)..(26): n is EdU
  misc_feature (28)..(29): n is EdU
agagacncacgggcgggcgaccnacngnncagcccagaccga

SEQ ID NO: 10 - DNA, 84 bp, Artificial
  aptamer
  misc_feature (22)..(63): N is a, c, g or EdU
cacgacgcaagggaccacaggnnnnnnnnnnnnnnnnnnnnnnnnnnnnnnnnnnnnnnnnnncagcacgacaccgcagaggca

SEQ ID NO: 11 - DNA, 21 bp, Artificial
  primer
cacgacgcaagggaccacagg

SEQ ID NO: 12 - DNA, 21 bp, Artificial
  primer
cacgacgcaagggaccacagg

SEQ ID NO: 13 - DNA, 21 bp, Artificial
  primer
tgcctctgcggtgtcgtgctg

SEQ ID NO: 14 - DNA, 84 bp, Artificial
  aptamer
  misc_feature (29)..(29): n is T or EdU
  misc_feature (36)..(36): n is T or EdU
  misc_feature (45)..(45): n is T or EdU
  misc_feature (54)..(54): n is T or EdU
cacgacgcaagggaccacaggaggagagnaggcganacacgacgnagcgcaganaggaccaagcagcacgacaccgcagaggca

SEQ ID NO: 15 - DNA, 84 bp, Artificial
  aptamer
  misc_feature (27)..(27): n is EdU
  misc_feature (37)..(37): n is EdU
  misc_feature (41)..(41): n is EdU
  misc_feature (48)..(48): n is EdU
  misc_feature (59)..(59): n is EdU
cacgacgcaagggaccacagggcacgngcgaggcgcnacancaccggngcaagcaccgngcaacagcacgacaccgcagaggca

SEQ ID NO: 16 - DNA, 15 bp, Artificial
  aptamer
  misc_feature (5)..(5): n is a, c, g, t or EdU
  misc_feature (6)..(6): n is EdU
  misc_feature (9)..(9): n is EdU
  misc_feature (11)..(12): n is EdU
cgacnnacngnncag

SEQ ID NO: 17 - DNA, 43 bp, Artificial
  click competitor
  misc_feature (1)..(42): n is a, c, g or EdU
nnnnnnnnnnnnnnnnnnnnnnnnnnnnnnnnnnnnnnnnnna

(19) United States
(12) Patent Application Publication
(10) Pub. No.: US 2023/0358760 A1
(43) Pub. Date: Nov. 9, 2023

(54) APTAMERS THAT TARGET CXCL9

(71) Applicants: RHEINISCHE FRIEDRICH-WILHELMS-UNIVERSITÄT BONN, Bonn (DE); LEIBNIZ UNIVERSITÄT HANNOVER, Hannover (DE)

(72) Inventors: Günter MAYER, Bonn (DE); Julia SIEGL, Bonn (DE); Cornelia BLUME, Wedemark (DE); Linh PHUNG, Hannover (DE)

(73) Assignees: RHEINISCHE FRIEDRICH-WILHELMS-UNIVERSITÄT BONN, Bonn (DE); LEIBNIZ UNIVERSITÄT HANNOVER, Hannover (DE)

(21) Appl. No.: 18/042,882

(22) PCT Filed: Aug. 31, 2021
(86) PCT No.: PCT/EP2021/074003

§ 371 Date: Feb. 24, 2023

(30) Foreign Application Priority Data
Aug. 31, 2020 (EP) 20193726.5

(51) Int. Cl. G01N 33/68; G01N 33/53; G01N 33/543

(52) U.S. Cl. CPC G01N 33/6869; G01N 33/5308; G01N 33/54326; G01N 33/54388; G01N 2333/5425; G01N 2800/245

(57) ABSTRACT

The present invention relates to an aptamer comprising a nucleotide sequence SEQ ID NO: 1, preferably comprising or consisting of a nucleotide sequence SEQ ID NO: 4-6. The invention further relates to a composition comprising the aptamer, and the use of the aptamer as a medicament or a diagnostic reagent, particularly for use in the detection or diagnosing of a rejection of a renal allograft.

The present invention relates to aptamers that target CXCL9. These aptamers are particularly useful in the detection of renal transplant rejection.

Kidney transplantation is a curative and life-saving therapy for end-stage renal disease which has become a standard in industrialised countries. The major limiting factor for transplant survival and success of kidney transplantation is transplant rejection. Rejection of renal allografts is a common poster-surgery risk factor. If diagnosed early, the successful treatment is promising. The standard for diagnosing transplant rejection is made by biopsy. As the procedure of renal transplant biopsies is afflicted with serious clinical risks and side effects it cannot be applied frequently in clinical routine. Therefore numerous approaches for non-invasive diagnostic strategies such as the screening of proteins as markers for detection of transplant rejection have been established.

The pro-inflammatory chemokine receptor ligand 9 CXCL9 (CXCL 9) is one early onset marker for the rejection of the donor organ. In search for highly sensitive diagnostic tools which can be used as an early POC (point of care) test, aptamers in contrast to most antibodies have a high thermostability and no batch-to-batch variations and thus are an interesting tool for diagnostic tests. For example Ferguson et al., Sci Transl Med. 2013, 5(213), 213ra165, suggest to use an aptamer-based detector for monitoring levels of the chemokine CXCL9, which is associated with nephropathy, acute graft rejection and graft failure, to enhance therapeutic outcome for patients undergoing organ transplantation.

Aptamers are single chained nucleic acids, folding into well-defined three-dimensional shapes based on which they recognise target structures with high affinity and specificity. Aptamers of either single-stranded DNA or RNA that specifically bind to a target ligand are produced by a technique denoted Systematic Evolution of Ligands by Exponential Enrichment (SELEX). The SELEX method is for example described by C. Tuerk and L. Gold “Systematic Evolution of Ligands by Exponential Enrichment: RNA Ligands to bacteriophage T4 DNA polymerase”, Science 1990, 249, 505-510. In the SELEX method, a candidate mixture of single stranded nucleic acids having regions of randomized sequence is contacted with a target structure and those nucleic acids having an increased affinity to the target are selected and amplified. After several iterations a nucleic acid with high affinity to the target is obtained. One drawback of the selection of conventional aptamers is their limited chemical diversity, hence, targeting of difficult molecules can be challenging. Further, the selection of aptamers that are chemically modified via so-called multimodal click-SELEX (Systematic Evolution of Ligands by Exponential Enrichment) is known.

Therefore, the object underlying the present invention was to provide aptamers that are able to target CXCL9.

The problem is solved by an aptamer comprising a nucleotide sequence 5′-N₁N₂GN₃CCN₁AN₄N₅N₁N₆N₇N₈N₁-3′ (SEQ ID NO: 1) or pharmaceutically acceptable salts thereof, wherein:
N₁ represents T or 5-ethynyl-2′-deoxyuridine (EdU) which is modified with a side chain selected from the group comprising indole, benzofurane, naphthalene, phenol, benzothiophene, guanidine and benzyl,
N₂ represents a sequence of 9 or 11 contiguous nucleotides comprising at least 5 guanidine nucleotides,
N₃ represents A or C,
N₄ represents A or a deletion,
N₅ represents C or G,
N₆ represents C or a deletion,
N₇ represents C or G,
N₈ represents G or N₁.

Surprisingly, aptamers could be selected that recognise the chemokines CXCL9 and CXCL11 with high affinity and specificity. The aptamers further were shown to be able to bind to the recombinant and to the endogenous CXCL9. The aptamers thus are usable to detect CXCL9 in biological samples. The chemokine CXCL9 is an early onset marker for organ rejection in plasma and urine of kidney transplant patients. The aptamers providing high affinity and specificity for CXCL9 and CXCL11 thus allow a use as non-invasive biomarkers for diagnosing transplant rejection in kidney transplant recipients. The aptamers are usable in diagnostic tests for the detection of a kidney rejection. Such tests can improve early diagnosis-making in transplant rejection after kidney transplantation and help predict long-term outcome after kidney transplantation. Generally, aptamer represents a cost-effective tool as aptamers can be synthesised chemically.

As used herein, the term “aptamer” refers to a single-stranded oligonucleotide that recognises its target with high specificity and binds to the target with high affinity in the low nanomolar range. The aptamer can be provided in the form of a single-stranded DNA or RNA molecule. As will be obvious to a person of ordinary skills in the art, if the nucleic acid is an RNA molecule the thymidine or “T” in the nucleotide sequence is to be read as meaning “U” or uridine. Preferably, the aptamer comprises a deoxyribonucleotide sequence. DNA aptamers can exhibit better stability. The nucleotides may comprise a chemical modification such as an aromatic group.

The aptamers comprise a G-rich sequence comprising the sequence of 9 or 11 contiguous nucleotides N₂, followed by a shorter motif comprising three or four thymidines (Ts) or modified 5-ethynyl-2′-deoxyuridines (EdUs) that were found to be important for binding to CXCL9. The G-rich sequence N₂ comprises at least 5 further guanidine nucleotides. In case N₂ represents a sequence of 9 contiguous nucleotides N₂ may comprise 5 further guanidine nucleotides. In case N₂ represents a sequence of 11 contiguous nucleotides N₂ may comprise 6 further guanidine nucleotides. Preferably, the aptamer comprises two triplicates of guanidines, either comprised in contiguous nucleotides of N₂ or including the G following N₂. The further nucleotides of the sequence of 9 or 11 contiguous nucleotides of N₂ may be selected from A, C, G or T, preferably from A and C.

The aptamer may comprise a nucleotide sequence 5′-NiGGGCAAAGGGCCCN₁AAGN₁ CCGN₁-3′ (SEQ ID NO: 2) or pharmaceutically acceptable salts thereof, wherein N₁ represents T or EdU which is modified with a side chain selected from the group comprising indole, phenol, guanidine and benzyl. Or the aptamer may comprise a nucleotide sequence 5′-N₁CACGGGCGGGCGACCN₁ACN₁GN₁N₁-3′ (SEQ ID NO: 3) wherein N₁ represents T or EdU which is modified with an aromatic group selected from the group comprising indole, benzofurane, naphthalene, phenol and benzothiophene.

EdU (5-ethynyl-2′-deoxyuridine) is a nucleoside analog of thymidine, carrying an ethynyl group. EdU is incorporated into replicating DNA instead of its natural analog thymidine. The ethynyl groups of the resulting ethynyl-functionalized DNA can subsequently be modified via Cu(I)-catalyzed click chemistry with azides of various compounds, such as with a side chain selected from indole, benzofurane, naphthalene, phenol or benzothiophene benzyl, or guanidine. In embodiments, the aptamer comprises or consists of a nucleotide sequence 5′-CACGACGCAAGGGACCACAGGGAGGGAGGGN₁GGGCAAAGGGCCCN₁A AGN₁CCGN₁AACAAAAACACAGCACGACACCGCAGAGGCA-3′ (SEQ ID NO: 4) or pharmaceutically acceptable salts thereof, wherein N₁ represents T or EdU which is modified with a side chain selected from the group comprising indole, phenol, guanidine and benzyl. The aptamers were shown to recognise the chemokines CXCL9 and CXCL11 with high affinity and specificity. The aptamers of the sequence of SEQ ID NO: 4 were shown to provide similar affinity for CXCL9 and CXCL11, but no binding to CXCL10.

The aptamer of SEQ ID NO: 4 was shown to be able to bind to CXCL9 and CXCL11 either as non-modified, conventional DNA or modified with different side chains. The aptamer of SEQ ID NO: 4 thus may comprise EdU moieties modified with side chains selected from indole, phenol, guanidine or benzyl or may be used as conventional DNA. In preferred embodiments, the aptamer comprises or consists of a nucleotide sequence 5′-CACGACGCAAGGGACCACAGGGAGGGAGGGTGGGCAAAGGGCCCTA AGTCCGTAACAAAAACACAGCACGACACCGCAGAGGCA-3′ (SEQ ID NO: 6) or of SEQ ID NO: 4 wherein N₁ represents EdU which is modified with with side chains selected from indole, phenol, guanidine or benzyl, or pharmaceutically acceptable salts thereof. In preferred embodiments, the aptamer comprises or consists of a nucleotide sequence SEQ ID NO: 4 or pharmaceutically acceptable salts thereof, wherein N₁ represents EdU which is modified with indole. The aptamer of SEQ ID NO: 4 modified with indole residues was shown to provide enhanced binding abilities for CXCL9.

In further embodiments, the aptamer comprises or consists of a nucleotide sequence 5′-CACGACGCAAGGGACCACAGGAGAGACN₁CACGGGCGGGCGACCN′₁ACN′₁ GN′₁N′₁CAGCCCAGACCGACAGCACGACACCGCAGAGGCA-3′ (SEQ ID NO: 5) or pharmaceutically acceptable salts thereof, wherein N₁ represents T or EdU which is modified with an aromatic group selected from the group comprising indole, benzofurane, naphthalene, phenol and benzothiophene and N′₁ represents EdU which is modified with an aromatic group selected from the group comprising indole, benzofurane, naphthalene, phenol and benzothiophene. The aptamer of SEQ ID NO: 5 was shown to bind to CXCL9 when modified with aromatic groups selected from indole, benzofurane, naphthalene, phenol or benzothiophene. The aptamer was shown to recognise the chemokine CXCL9 with high affinity and specificity, but no binding to CXCL10 was detected. In preferred embodiments, the aptamer comprising or consisting of a nucleotide sequence SEQ ID NO: 5 or pharmaceutically acceptable salts thereof is modified with an aromatic group selected from indole, benzofurane or naphthalene. Indole, benzofurane and naphthalene modifications provided enhanced binding to chemokine CXCL9.

The first N₁ member of SEQ ID NO: 5 may be selected from T or modified EdU. In embodiments, N₁ and N′₁ both represent EdU modified with an aromatic group. In embodiments, the aptamer may comprise or consist of a nucleotide sequence 5′-CACGACGCAAGGGACCACAGGAGAGACTCACGGGCGGGCGACCN′₁ACN′₁ GN′₁N′₁CAGCCCAGACCGACAGCACGACACCGCAGAGGCA-3′ (SEQ ID NO: 7) or pharmaceutically acceptable salts thereof, wherein N′₁ represents EdU which is modified with an aromatic group selected from the group comprising indole, benzofurane, naphthalene, phenol and benzothiophene.

The aptamer further may comprise or consist of a nucleotide sequence 5′-GAGGGNiGGGCAAAGGGCCCN₁AAGNiCCGN₁AACAAAAACA-3′ (SEQ ID NO: 8) or pharmaceutically acceptable salts thereof, wherein N₁ represents T or EdU which is modified with a side chain selected from the group comprising indole, phenol, guanidine and benzyl. Or the aptamer may comprise or consist of a nucleotide sequence 5′-AGAGACN₁CA CGGGCGGGCGACCN′₁ACN′₁GN′₁N′₁CAGCCCAGACCGA-3′ (SEQ ID NO: 9) or pharmaceutically acceptable salts thereof, wherein N₁ represents T or EdU which is modified with an aromatic group selected from the group comprising indole, benzofurane, naphthalene, phenol and benzothiophene, and N′1 represents EdU which is modified with an aromatic group selected from the group comprising indole, benzofurane, naphthalene, phenol and benzothiophene.

The aptamers also are usable in form of pharmaceutically acceptable salts thereof. The term “pharmaceutically acceptable salts” refers to salts prepared from pharmaceutically acceptable non-toxic bases or acids. A pharmaceutically acceptable salt can be conveniently prepared from pharmaceutically acceptable non-toxic bases, including inorganic bases and organic bases. Preferred salts derived from inorganic bases include ammonium, calcium, magnesium, potassium and sodium salts. Salts derived from pharmaceutically acceptable organic non-toxic bases include salts of primary, secondary, and tertiary amines, as well as cyclic amines. Preferably, the pharmaceutically acceptable salt is selected from the group of sodium or potassium salts. Also, calcium or magnesium salts can be preferred.

A further aspect relates to an aptamer comprising or consisting of a nucleotide sequence selected from the group of SEQ ID NO: 1-9, preferably from SEQ ID NO: 4-6, or pharmaceutically acceptable salts thereof for use as a medicament or a diagnostic reagent. For the description of the aptamers, reference is made to the description above. In preferred embodiments, the aptamer comprises or consists of a nucleotide sequence SEQ ID NO: 6 or SEQ ID NO: 4 wherein N₁ represents EdU which is modified with indole, phenol, guanidine and benzyl, preferably indole, or pharmaceutically acceptable salts thereof. In further preferred embodiments, the aptamer comprises or consists of a nucleotide sequence 5′-CACGACGCAAGGGACCACAGGAGAGACN₁CACGGGCGGGCGACCN′₁ACN′₁ GN′₁N′₁CAGCCCAGACCGACAGCACGACACCGCAGAGGCA-3′ (SEQ ID NO: 5) or pharmaceutically acceptable salts thereof, wherein N₁ represents T or EdU which is modified with an aromatic group selected from the group comprising indole, benzofurane, naphthalene, phenol and benzothiophene and N′₁ represents EdU which is modified with an aromatic group selected from the group comprising indole, benzofurane, naphthalene, phenol and benzothiophene.

The aptamers advantageously are able to recognise chemokines CXCL9 with high affinity and in biological samples. This allows that the aptamer can be used for the detection of early onset marker for the rejection of a transplanted organ and suggests a use in the diagnosis of transplant rejection. The aptamers thus allow a use as non-invasive biomarkers for diagnosing transplant rejection in kidney transplant recipients. The aptamers particularly are usable as a diagnostic reagent in tests for the detection of a kidney rejection. Hence, the aptamer is useful as a medicament or a diagnostic reagent.

In preferred embodiments, the aptamer is for use in the detection or diagnosing of a rejection of a renal allograft. The aptamer allows detecting binding to chemokines CXCL9 with high affinity and specificity in biological samples and thus a use in highly sensitive diagnostic tools to be used as an early POC (point of care) test. The aptamers may help to improve early diagnosis-making in intransplant rejection after kidney transplanation.

The aptamer may be used alone or in combination with further markers for pro-inflammatory chemokines such as antibodies. The aptamer may be bound or forming a complex, such as a sandwich complex with antibodies. The aptamer may be used with antibodies detecting CXCL9, or antibodies detecting further chemokines. In preferred embodiments, the aptamer is used in form a complex with an antibody against chemokines CXCL9. By detecting binding to chemokine CXCL9 via aptamer and antibody specificity of a test may be optimised.

For use as a medicament or a diagnostic reagent the aptamers can be used or included in a composition. For use as a medicament the aptamer can be used or included in a pharmaceutical composition.

For use as a diagnostic reagent the aptamer can be used or included in a diagnostic composition. The aptamers, for example, may be used as a diagnostic reagent in lateral flow assays or in enzyme-linked oligonucleotide assays (ELONA). Accordingly, a further aspect relates to a diagnostic composition comprising as an active ingredient an aptamer comprising or consisting of a nucleotide sequence selected from the group of SEQ ID NO: 1-9, preferably from SEQ ID NO: 4-6, or pharmaceutically acceptable salts thereof. A further aspect relates to a diagnostic test, particularly a lateral flow assay, more particularly a test strip, comprising as a diagnostic reagent an aptamer comprising or consisting of a nucleotide sequence selected from the group of SEQ ID NO: 1-9, preferably from SEQ ID NO: 4-6, or pharmaceutically acceptable salts thereof. For the description of the aptamers, reference is made to the description above. In preferred embodiments, the aptamer comprises or consists of a nucleotide sequence SEQ ID NO: 6 or SEQ ID NO: 4 wherein N₁ represents EdU which is modified with indole, phenol, guanidine and benzyl, preferably indole, or pharmaceutically acceptable salts thereof. In further preferred embodiments, the aptamer comprises or consists of a nucleotide sequence 5′-CACGACGCAAGGGACCACAG GAGAGACN₁CACGGGCGGGCGACCN′₁ACN′₁GN′₁N′₁CAGCCCAGACCGACAGCACG ACACCGCAGAGGCA-3′ (SEQ ID NO: 5) or pharmaceutically acceptable salts thereof, wherein N₁ represents T or EdU which is modified with an aromatic group selected from the group comprising indole, benzofurane, naphthalene, phenol and benzothiophene and N′₁ represents EdU which is modified with an aromatic group selected from the group comprising indole, benzofurane, naphthalene, phenol and benzothiophene.

By detecting binding to chemokines CXCL9 and CXCL11, the composition particularly is usable in the detection or diagnosis of the rejection of the donor organ such as after kidney transplanation. In preferred embodiments, the diagnostic composition is for use in the detection or diagnosing of a rejection of a renal allograft. The diagnostic composition comprising the aptamers may help to improve early diagnosis-making in intransplant rejection after kidney transplanation.

When the aptamer is brought into contact with a sample, it will bind specifically to chemokines CXCL9 and CXCL11 present in the sample. For diagnostic purposes, the aptamer may comprise a labelling which provides that the bound aptamer can be detected by determining the presence or absence of a signal provided by the label. For example, the aptamer can be labelled with a fluorescent dye. A fluorescence-labelling, for example provided by a fluorescence dye, can provide a visualisation of the bound aptamer by fluorescence or laser scanning microscopy or flow cytometry. Further, particularly for detection purposes, the aptamer can be immobilised on conventional supports such as beads providing a tool for the detection of aptamer-bound chemokine and thus diagnosing possible rejection of renal allograft. Further, the aptamer can be biotinylated or coupled to streptavidin, avidin or neutravidin for use in the specific detection of chemokines.

Besides being useful for detecting or diagnosing rejection of a donor organ by recognising chemokines, the aptamers also are applicable for targeted therapies. Particularly for use as a medicament the aptamers can be used or included in a pharmaceutical composition. Accordingly, in another aspect the present invention relates to a pharmaceutical composition comprising as an active ingredient an aptamer comprising or consisting of a nucleotide sequence selected from the group of SEQ ID NO: 1-9, preferably from SEQ ID NO: 4-6, or pharmaceutically acceptable salts thereof. For the description of the aptamers, reference is made to the description above. In preferred embodiments, the aptamer comprises or consists of a nucleotide sequence SEQ ID NO: 6 or SEQ ID NO: 4 wherein N₁ represents EdU which is modified with indole, phenol, guanidine and benzyl, preferably indole, or pharmaceutically acceptable salts thereof. In further preferred embodiments, the aptamer comprises or consists of a nucleotide sequence 5′-CACGACGCAAGGGACCACAGGAGAGACN₁CACGGGC GGGCGACCN′₁ACN′₁GN′₁N′₁CAGCCCAGACCGACAGCACGACACCGCAGAGGCA-3′ (SEQ ID NO: 5) or pharmaceutically acceptable salts thereof, wherein N₁ represents T or EdU which is modified with an aromatic group selected from the group comprising indole, benzofurane, naphthalene, phenol and benzothiophene and N′₁ represents EdU which is modified with an aromatic group selected from the group comprising indole, benzofurane, naphthalene, phenol and benzothiophene.

The pharmaceutical composition comprising the aptamers can particularly be useful as a molecular vehicle for delivery of cargo such as anti-inflammatory agents to the chemokines for reducing inflammation and donor organ rejection. Compounds, drugs or effector molecules can be directly coupled to the aptamer, in a covalent or non-covalent fashion. Alternatively, the aptamer can be attached to the surface of a liposome containing an anti-inflammatory agent or to nanoparticles encapsulating an anti-inflammatory agent. The aptamers may provide a chemokine-specific drug delivery composition comprising an aptamer and an anti-inflammatory agent such as an anti-inflammatory drug.

The composition may be produced under sterile conditions using standard pharmaceutical techniques well known to those skilled in the art. For compositions convenient pharmaceutical media may be employed. For example, water, glycols, oils, alcohols and the like may be used to form liquid preparations such as solutions. The composition may comprise a pharmaceutical carrier, which can be, for example, a solid, liquid, or gas. Suitable carriers preferably are liquid and correspond to the substances ordinarily employed in formulation technology for pharmaceutical formulations.

The present invention also relates to the use of an aptamer comprising or consisting of a nucleotide sequence selected from the group of SEQ ID NO: 1-9, preferably from SEQ ID NO: 4-6, or pharmaceutically acceptable salts thereof, for the manufacture of a medicament or a diagnostic reagent. The diagnostic reagent particularly is for use in the detection or diagnosing of a rejection of a renal allograft. For the description of the aptamers, reference is made to the description above. In preferred embodiments, the aptamer comprises or consists of a nucleotide sequence SEQ ID NO: 6 or SEQ ID NO: 4 wherein N₁ represents EdU which is modified with indole, phenol, guanidine and benzyl, preferably indole, or pharmaceutically acceptable salts thereof. In further preferred embodiments, the aptamer comprises or consists of a nucleotide sequence 5′-CACGACGCAAGGGACCACAGGAGAGACN₁CACGGGC GGGCGACCN′₁ACN′₁GN′₁N′₁CAGCCCAGACCGACAGCACGACACCGCAGAGGCA-3′ (SEQ ID NO: 5) or pharmaceutically acceptable salts thereof, wherein N₁ represents T or EdU which is modified with an aromatic group selected from the group comprising indole, benzofurane, naphthalene, phenol and benzothiophene and N′1 represents EdU which is modified with an aromatic group selected from the group comprising indole, benzofurane, naphthalene, phenol and benzothiophene.

A further aspect relates to an in vitro method of detecting or diagnosing a rejection of a renal allograft, the method comprising the step of detecting the binding of an aptamer comprising or consisting of a nucleotide sequence selected from the group of SEQ ID NO: 1-9, preferably from SEQ ID NO: 4-6, or pharmaceutically acceptable salts thereof, to CXCL9 or CXCL11 in a sample obtained from a subject. For the description of the aptamers, reference is made to the description above. In preferred embodiments, the aptamer comprises or consists of a nucleotide sequence SEQ ID NO: 6 or SEQ ID NO: 4 wherein N₁ represents EdU which is modified with indole, phenol, guanidine and benzyl, preferably indole, or pharmaceutically acceptable salts thereof. In further preferred embodiments, the aptamer comprises or consists of a nucleotide sequence 5′-CACGACGCAAGGGACCACAGGAGAGACN₁CACGGGCGGGCGACCN′₁ACN′₁ GN′₁N′₁CAGCCCAGACCGACAGCACGACACCGCAGAGGCA-3′ (SEQ ID NO: 5) or pharmaceutically acceptable salts thereof, wherein N₁ represents T or EdU which is modified with an aromatic group selected from the group comprising indole, benzofurane, naphthalene, phenol and benzothiophene and N′₁ represents EdU which is modified with an aromatic group selected from the group comprising indole, benzofurane, naphthalene, phenol and benzothiophene.

As used herein, the term “sample” refers to any material, which probably contains chemokines, particularly CXCL9 or CXCL11, including any liquid or fluid sample or solid material, particularly a sample derived from a biological source such as a patient or test subject. The sample may be derived from a biological source such as a renal allograft subject. The term sample particularly refers to biological material, for example cells or tissues, biological fluids or supernatants. The sample for example can comprise cells or a tissue specimen isolated from a cancer subject, preferably a human, for example, by surgical resection or biopsy. The sample can be a body fluid such as blood, serum, plasma, saliva, phlegm and urine. The sample preferably is selected from urine or serum of a renal allograft patient, preferably a human. The method comprises bringing the aptamer into contact with a sample, which probably contains chemokines, particularly CXCL9 or CXCL11. Determination of biomarkers in urine samples is commonly used for point of care testing.

Unless otherwise defined, the technical and scientific terms used herein have the same meaning as commonly understood by one of ordinary skill in the art to which this invention belongs.

The Examples, which follow serve to illustrate the invention in more detail but do not constitute a limitation thereof.

The figures show:

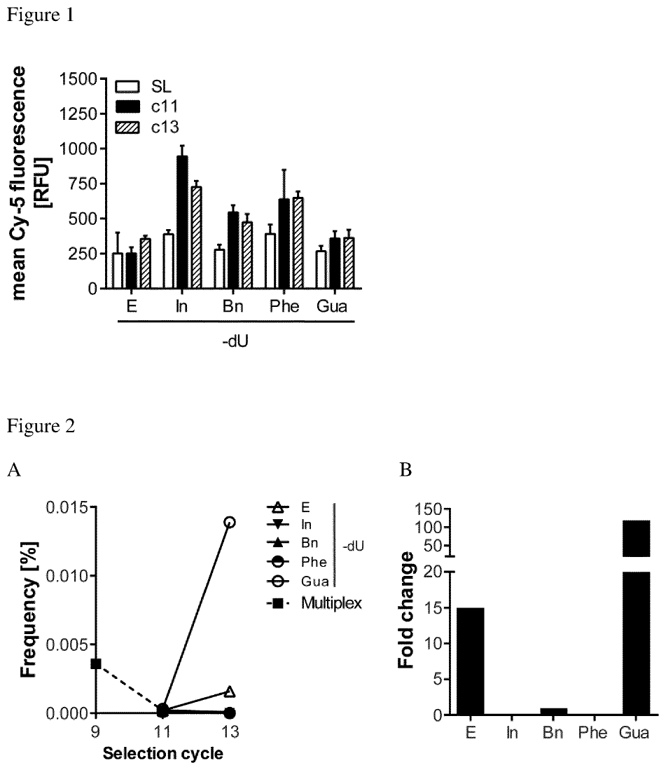

FIG. 1 Interaction analysis using flow cytometry of a starting library binding to CXCL9 immobilised on beads and after 11 or 13 selection cycles. 500 nM Cy-5 labelled DNA from the starting library (SL) as well as from selection cycle 11 and selection cycle 13 were incubated with CXCL9 immobilised on magnetic beads. DNA was either unmodified (E-dU) or click modified with indole (In), benzyl (Bn), phenol (Phe) or guanidine (Gua) azide.

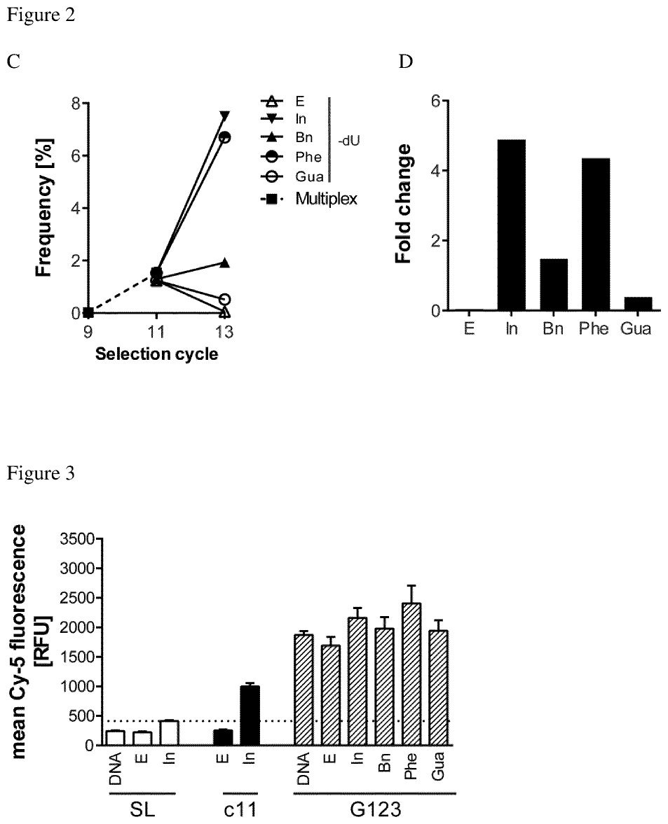

FIG. 2 in FIG. 2A the frequency and in FIG. 2B fold change of selection cycle 11 and selection cycle 13 of sequence SEQ ID NO: 4 (G123), and in FIG. 2C the frequency and and in FIG. 2D fold change of selection cycle 11 and selection cycle 13 of sequence SEQ ID NO: 5 (I29).

FIG. 3 Interaction analysis with flow cytometry. 500 nM Cy-5 labelled DNA from the starting library (SL), from selection cycle 11 (c11) and sequence SEQ ID NO: 4 (G123) were incubated with CXCL9 immobilised on magnetic beads. DNA was either conventional DNA, unmodified (E-dU) or click modified with indole (In), benzyl (Bn), phenol (Phe) or guanidine (Gua) azide.

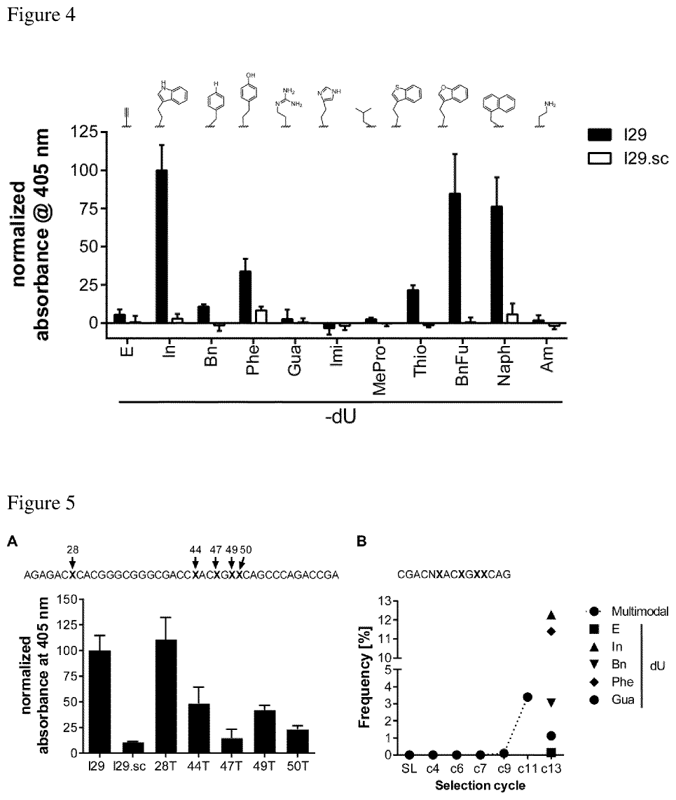

FIG. 4 Influence of modification for aptamer SEQ ID NO: 5 (I29) binding to CXCL9. Biotin labelled DNA was detected by colorimetric change using streptavidin-horseradish peroxidase (SA-HRP) A binding of 100 nM of aptamer SEQ ID NO: 5 (I29) with 10 different modifications (or w/o=E) to CXCL9.

FIG. 5 Substitution of the EdU modifications with conventional Ts of aptamer SEQ ID NO: 5 (I29). Biotin labelled DNA (100 nM) of 129 variants was detected by colorimetric change using streptavidin-horseradish peroxidase (SA-HRP).

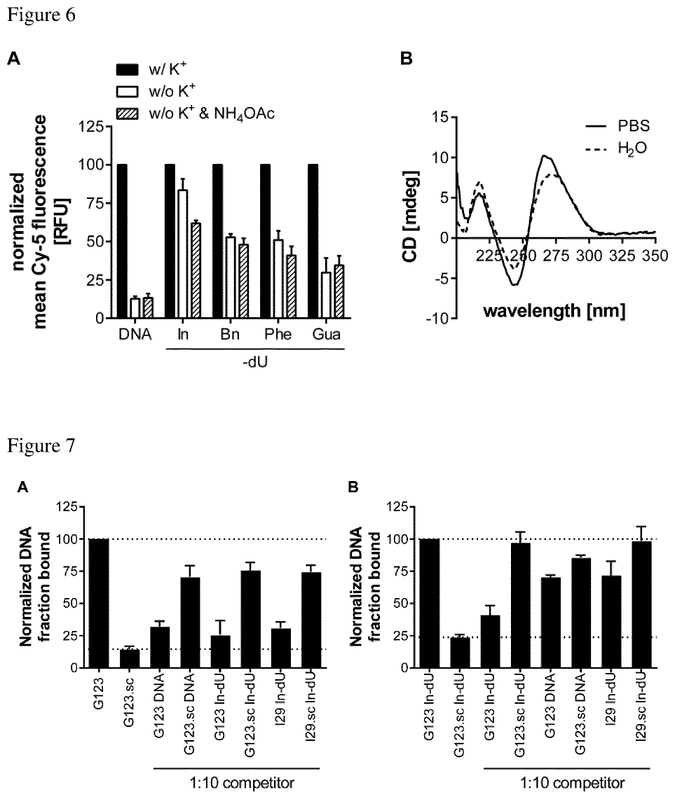

FIG. 6 G-quadruplex characterisation of G123 sequence. FIG. 6A shows results of a flow cytometer binding assay. FIG. 6B shows a circular dichroism analysis of SEQ ID NO: 4 (G123) DNA in water and in PBS.

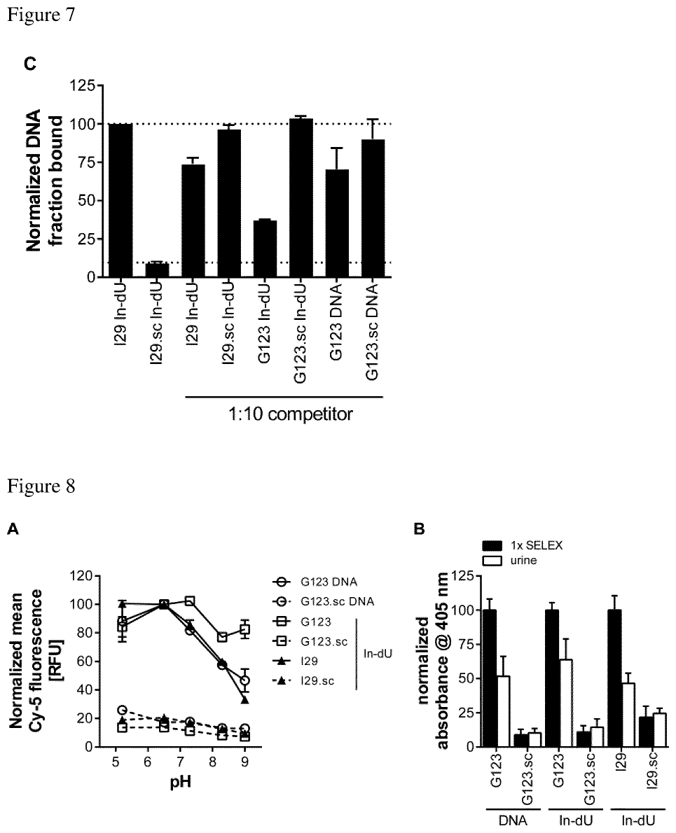

FIG. 7 Competition assay of the indole-modified aptamer of SEQ ID NO: 5 (I29 In), unmodified aptamer of SEQ ID NO: 6 (G123 DNA), and indole-modified aptamer of SEQ ID NO: 4 (G123 In).

FIG. 8 Characterisation of CXCL9 binding in different buffer conditions. FIG. 8A shows results of a flow cytometer binding assay of conventional aptamer of SEQ ID NO: 6 (G123 DNA), the aptamer of SEQ ID NO: 4 click-modified with indole azide (G123In-dU), and the aptamer of SEQ ID NO: 5 click-modified with indole azide (G123In-dU) (n=3-4, SD±mean, binding normalized to binding in SELEX conditions (pH 6.5)). FIG. 8B shows results of an ELONA binding assay of biotin labelled G123 (DNA or with indole (In) modification) and I29 (In) in either SELEX conditions or in human urine.

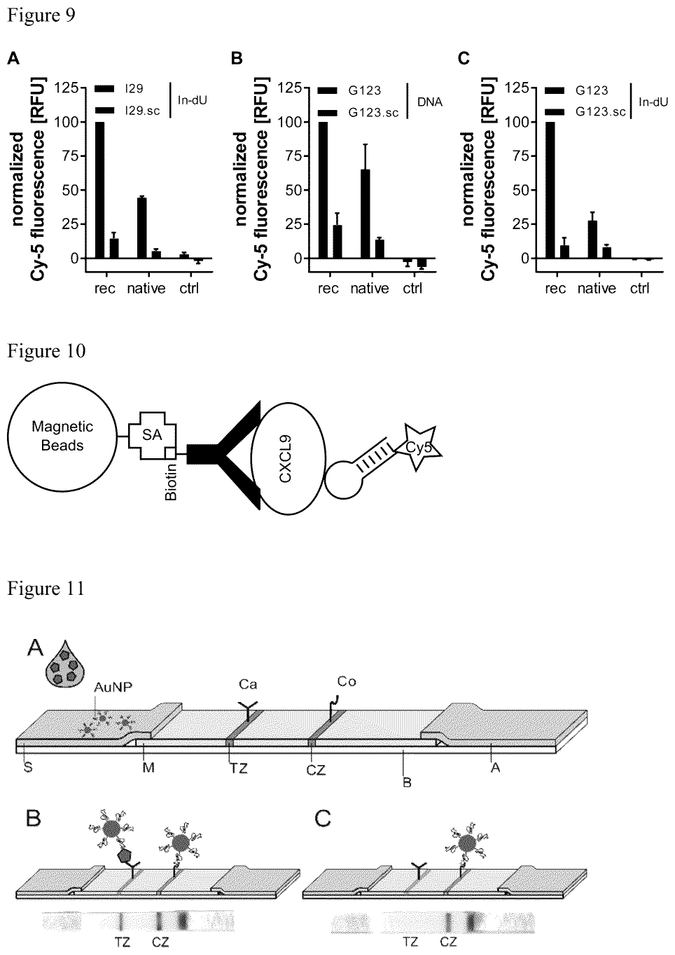

FIG. 9 Binding to endogenous CXCL9. Binding to native CXCL9 in cell supernatant (native), recombinant CXCL9 in control cell supernatant (rec) and control supernatant without CXCL9 (ctrl) with Cy5-labelled aptamer of SEQ ID NO: 5 click-modified with indole azide (I29) is shown in FIG. 9A, aptamer of SEQ ID NO: 6 (G123 DNA) in FIG. 9B and aptamer of SEQ ID NO: 4 click-modified with indole azide (G123 In) in FIG. 9C.

FIG. 10 sandwich complex

FIG. 11 Schematic view of the basic structure of an aptamer-antibody-hybrid-lateral flow assay showing sample pad (S), nitrocellulose membrane (M), backing card (B) absorbent pad (A), test zone (TZ), control zone (CZ), AuNP-labeled detection aptamer (AuNP), capture antibody against CXCL9 (Ca), and capture oligonucleotide against the detection aptamer (Co).

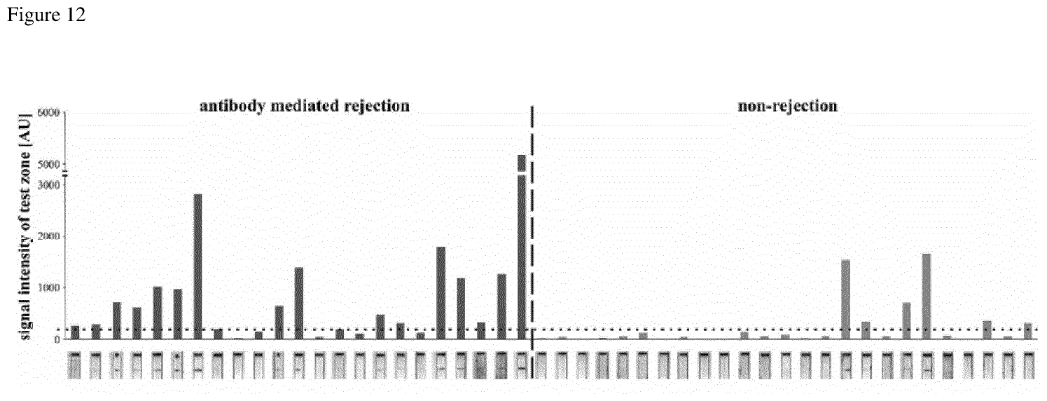

FIG. 12 Signal intensity of the test line of 48 lateral flow assay strips tested with patient urine samples showing the test and control zone.

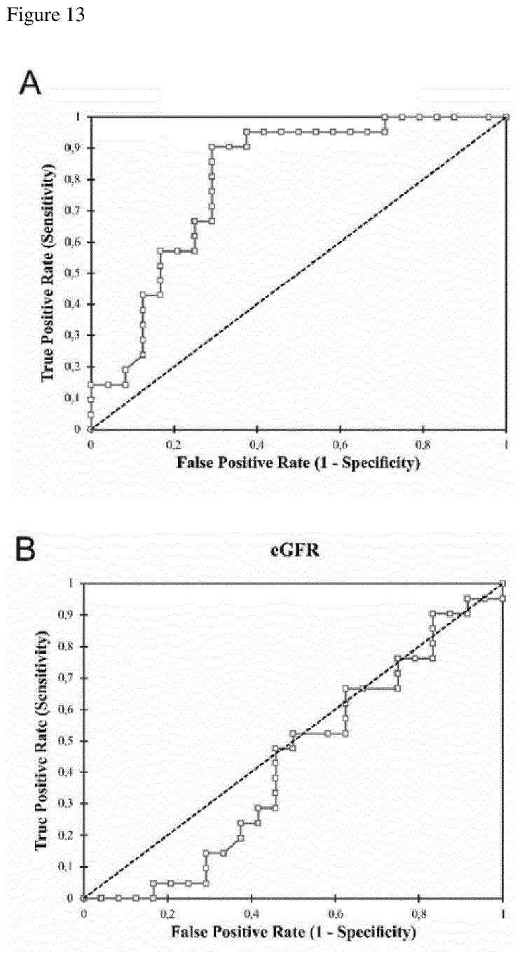

FIG. 13 Area under the curve (AUC) values in receiver operating characteristic (ROC) analyses of A) hybrid LFA (AUC=0.799) and B) estimated glomerular filtration rate (eGFR, AUC=0.428) at day of biopsy.

### REAGENTS AND MATERIALS

### Chemicals

All chemicals, unless otherwise stated, where purchased from Sigma-Aldrich (Munich, Germany). 5-Ethynyl-dUTP (EdU) and tris(3-hydroxypropyltriazolmethyl)amine (THPTA) were purchased from BaseClick (Neuried, Germany). M280 streptavidin magnetic beads, salmon sperm DNA, EZ-Link Sulfo-NHS-LC-Biotin and λ-exonuclease were obtained from Thermo Fisher Scientific (Darmstadt, Germany). Preparations of azides (indole, phenole, guanidine) was done according to the protocol described in Suzuki et al., “Rapid discovery of highly potent and selective inhibitors of histone deacetylase 8 using click chemistry to generate candidate libraries”, J Med Chem, 2012. 55 (22): p. 9562-75. Benzyl azide was purchased from Sigma Aldrich.

### Proteins and Enzymes

Proteins and enzymes were purchased from the suppliers as given in the following table 1:

TABLE 1
Proteins and enzymes:

| Protein | Supplier |
|---|---|
| Human CXCL9 | Origene |
| BSA | AppliChem |
| Human CXCL10 | Cell guidance system |
| Human CXCL11 | Cell guidance system |
| Human CXCL1 | Cell guidance system |
| Human sCD25 | Antikorper online |
| Native human CRP | BioRad |
| Human CCL17 | BioLegend |
| Human CCL22 | BioLegend |
| Human CCL3 | Peprotech |
| HSA | Sigma-Aldrich |
| Human CXCL9/MIG Biotinylated Antibody | R&D System |
| PWO DNA Polymerase | Genaxxon |
| 2 exonuclease | Thermo Fisher Scientific |

### Oligonucleotides

HPLC purified oligonucleotides were purchased from Ella Biotech (Planegg, Germany). Sequences of the oligonucleotides are given in the following table 2:

TABLE 2
Sequences of oligonucleotides:

| Oligo Name | Sequence |
|---|---|
| FT2-0.35 | CACGACGCAAGGGACCACAGG-N42- |
| | CAGCACGACACCGCAGAGGCA |
| | (SEQ ID NO: 10) |
| | N = A:G:C:EdU (1:1:1:0.35) |
| Click | N42-A (SEQ ID NO: 17) |
| competitor | N = A:G:C:EdU (1:1:1:1) |
| FT2-Fw-Cy5 | Cy5-CACGACGCAAGGGACCACAGG |
| | (SEQ ID NO: 11) |
| FT2-Fw-Bio | Bio-CACGACGCAAGGGACCACAGG |
| | (SEQ ID NO: 12) |
| FT2-Rev-P | Phos-TGCCTCTGCGGTGTCGTGCTG |
| | (SEQ ID NO: 13) |
| G123 | CACGACGCAAGGGACCACAGGGAGGGAGGGNGGG |
| | CAAAGGGCCCNAAGNCCGNAACAAAAACACAGCA |
| | AGAGGCA (SEQ ID NO: 4) |
| | CGACACCGC |
| | N = T/EdU |
| G123.sc | CACGACGCAAGGGACCACAGGAGGAGAGNAGGCGA |
| | NACACGACGNAGCGCAGANAGGACCAAGCAGCACG |
| | ACACCGCAGAGGCA (SEQ ID NO: 14) |
| | N = T/EdU |
| I29 | CACGACGCAAGGGACCACAGGAGAGACNCACGGGCG |
| | GGCGACCNACNGNNCAGCCCAGACCGACAGCACGAC |
| | ACCGCAGAGGCA (SEQ ID NO: 5) |
| | N = EdU |
| I29.sc | CACGACGCAAGGGACCACAGGGCACGNGCGAGGCGC |
| | NACANCACCGGNGCAAGCACCGNGCAACAGCACGAC |
| | ACCGCAGAGGCA (SEQ ID NO: 15) |
| | N = EdU |

### Methods

### Biotinylation:

500 μl CXCL9 (39 nmol) were mixed with 15.7 μl sulfo-NHS-LC-Biotin (156 nmol) and incubated for 30 min on ice followed by incubation for 25 min at RT. Afterwards biotinylated protein was purified using zeba spin desalting columns (Thermo Fisher Scientific) according to manufacturer instructions.

### Bead Preparation:

10 mg of M280-streptavidin magnetic dynabeads were washed three times with 1000 μl PBS and resuspended in 1000 μl PBS. 500 μl were used as empty beads and 50 μl of biotinylated CXCL9 (66 μM) was added to the remaining beads. After incubation at 25° C. and 1000 rpm the supernatant was discarded, and the beads were washed three times with 500 μL PBS. Supernatant of empty and CXCL9 beads was discarded and beads were resuspended with 1× SB1 (138 mM NaCl, 5 mM KCl, 1.5 mM KH₂PO₄, 8.1 mM Na₂HPO₄, 170 mM urea, 7 mM ammonium acetate, 1 mM MgCl₂, 1 mM CaCl₂), 1.2 mg/mL BSA, 0.1 mg/mL salmon sperm DNA, pH 6.5) and stored at 4° C.

### Click Reaction:

The click reaction was done as described by Pfeiffer et al., “Identification and characterization of nucleobase-modified aptamers by click-SELEX”, Nat Protoc, 2018. 13 (5): p. 1153-1180. Shortly, freshly prepared sodium ascorbate (25 mM), CuSO₄ (1 mM) and THPTA (4 mM) in 100 μL ddH₂O are incubated for 10-15 min (catalyst solution). Afterwards, EdU-DNA, was clicked in a solution containing 1 mM azide in DMSO (10% final), 1× phosphate buffer and 1× catalyst solution in a total of 100 μL ddH₂O. The mixture was incubated 15 min at 37° C. and 650 rpm. Afterwards the samples were purified using Nucleospin Gel and PCR clean-up kit from Macherey-Nagel (Duren, Germany) according to the manufacturer's instructions.

### Polymerase Chain Reaction (PCR):

PCR was performed in a veriti 96 well thermo cycler (Applied Biosystem). All PCRs contained 0.5 μM of both forward and reverse primer, 250 μM d*NTP mix (with EdU instead of T), PWO DNA polymerase (Genaxxon) and the supplied buffer (containing 2 mM MgS04). The samples were prepared on ice and the following cycling program was used Step 1: 2 min 95° C., Step 2 (denaturing): 30 s 92° C., Step 3 (annealing): 30 s 62° C., Step 4 (extension): 1 min 72° C., Step 5: 2 min 72° C., hold 10° C.; Step 2-4 were repeated).

### Split and Combine Click-SELEX:

For the first selection round, 125 pmol FT2-0.35 library were independently click modified with In-, Bn-, Phe and Gua-dU. Modified DNA was pooled together and incubated with 50 μl CXCL9-magnetic beads in 1× SB1 for 30 min at 25° C. and 1000 rpm. After washing with 1×SB1 the beads were incubated for 5 min at 95° C., supernatant was used as template for PCR in a total volume of 800 μL. Purified dsDNA was incubated with 3.5 μl λ-exonuclease (10 U/μl, ThermoFisher) in 1× λ-exonuclease buffer for 20 min at 37° C. and 650 rpm. Samples were purified using Nucleospin Gel and PCR clean-up kit from Macherey-Nagel according to the manufacturer's instructions. Finally, purified ssDNA was aliquoted to four samples which are, together with 125 pmol click competitor, individually click modified.

To increase the selection pressure several steps were modified: (1) addition of click competitor starting at cycle 2 (125 pmol for each azide), (2) increase in washing time and volume, (3) addition and increase of dextran sulfate from cycle 7, (4) decrease in incubation time, (5) reducing the number of magnetic beads (cycle 3-6), (6) reduction of biotinylated CXCL9 for immobilisation (⅕^{th} to 1/1000^{th} original CXCL9 concentration used for bead preparation).

### Flow Cytometry

The binding interaction of Cy5 labelled DNA to protein immobilized or captured on magnetic beads was investigated with a FACSCanto II (BD Bioscience). In total a minimum of 30′000 events was recorded and the Cy5-fluorescence in the APC-A channel was analysed.

### Binding Interaction of Selection Cycles and Single Sequences

500 nM of Cy5 labelled DNA and 1.5 μL CXCL9 beads in 10 μL SB1 were incubated for 30 min, 25° C. and 1000 rpm. Afterwards, the beads were washed shortly in 100 μL 1× SB1 and resuspended in 100 μL 1× SB1 for flow cytometer analysis.

### Competition Assay

100 nM Cy5 labelled DNA and 1 μM unlabelled DNA were incubated with 1 μL CXCL9 magnetic beads in 10 μL 1× SB1. After incubation (30 min, 25° C., 1000 rpm) the beads were washed 1× short and 1×3 min with 100 μL SB1 and resuspended in 100 μl 1×SB1 for flow cytometer analysis.

### Pull Down Native CXCL9

50 μg M280 streptavidin magnetic beads were washed 3 times with 40 μL PBS. 0.24 μg of biotinylated human CXCL9 antibody (R&D System) was incubated with 50 μg beads in 20 μL PBS for 40 min, 25° C. and 1000 rpm. The beads were washed three times with 40 μL PBS, resuspended in 10 μL PBS+1% BSA and used directly. Recombinant CXCL9 was diluted in control supernatant w/o CXCL9 to 11 ng/ml. 100 μl cell supernatant containing endogenous CXCL9 (11 ng/ml), control supernatant with and without CXCL9 were incubated with 1 μL of antibody beads for 30 min, 25° C. and 1000 rpm. After, beads were washed 1× short and 1×3 min with 100 μl PBS+1% BSA. 40 μL 150 nM Cy5-DNA in 1× SB1 were incubated with beads for 30 min, 25° C., 1000 rpm. Without washing, the bead complex is analysed by flow cytometry. Binding of 150 nM Cy-5 DNA to antibody beads was used as background and was thus subtracted from samples.

### Binding in Different Buffer Conditions

CXCL9 beads were 100 nM Cy5 labelled DNA and 1.5 μl CXCL9 beads were incubated in 40 μL either 1× SB1 (without ammonium acetate and urea) or in 1× SB1 w/o K⁺ (10 mM H₃PO₄+NaOH, 1.2 mg/ml BSA, 0.1 mg/ml salmon sperm DNA) for 30 min at 25° C. and 1000 rpm. Beads are directly analysed without washing steps. pH of SB1 was adjusted accordingly for binding at different pH values.

### Next-Generation Sequencing (NGS):

NGS samples were prepared as described in Tolle et al. “Preparation of SELEX Samples for Next-Generation Sequencing”, Methods Mol Biol, 2016. 1380: p. 77-84 and were measured on a Illumina HiSeq1500 platform. Shortly, PCR with index primers was done using canonical nucleotides, and thus replacing the site of modification with a T. PCR product was purified using Nucleospin Gel and PCR clean-up kit from Macherey-Nagel (Duren, Germany) according to the manufacturer's instructions. 83 ng of each purified DNA waa pooled, and adapter sequence was added by enzymatic ligation using TruSeq DNA PCR-Free Sample Preparation Kit LT (Illumina). After DNA agarose purification and Nucleospin Gel and PCR clean-up kit from Macherey-Nagel (Duren, Germany) clean up, the DNA was eluted in resuspension buffer (TruSeq DNA PCR-Free Sample Preparation Kit LT (Illumina)).

Prior sequencing the DNA was validated and quantified using KAPA library quantification kit (Sigma-Aldrich). Illumina sequencing was performed with 75 bp single end sequencing. Analysis of raw data was done using AptaNext software (Laura Lledo Byrant).

### (Enzyme Linked Oligonucleotide Assay) ELONA

20 μl of 1 μg/ml protein in bicarbonate/carbonate buffer pH 9.6 was coated overnight at 4° C. on 96 well half area plates (Greiner Bio). After washing three times with 100 μL WB1 (PBS+0.05% Tween 20), wells were blocked with 1% BSA in PBS for 2 h at RT with slight agitation. Empty wells were also blocked for later subtraction of background binding. Wells were washed once with 100 μL WB2 (138 mM NaCl, 5 mM KCl, 1.5 mM KH₂PO₄, 8.1 mM Na₂HPO₄, 170 mM urea, 7 mM ammonium acetate, 1 mM MgCl₂, 1 mM CaCl₂), pH 6.5).

DNA was diluted to respective concentration in SB1 and 20 μL is transferred onto the wells in duplicates (protein and BSA wells). After incubation of 30 min at RT and slight agitation, the wells were washed two times with 100 μL WB1 and are incubated with 20 μL of streptavidin-horseradish peroxidase (SA-HRP) solution (1:1000 in PBS, GE Healtcare). After 30 min incubation at RT the wells were washed again twice with 100 μL WB1. Finally, the complex was incubated with 1-Step ABTS (ThermoFisher) for 15-40 min at RT, slight agitation. Absorbance at 405 nm was read out with a Tecan Nanoquant plate reader. Binding of DNA to BSA background was subtracted from binding of DNA to protein sample.

The equilibrium dissociation constant was calculated with non-linear regression assuming one site specific binding model with GraphPad Prism 6.01 (Graph Pad Software, La Jolla, USA)

### Example 1

### Selection of Aptamers Using Multimodal Split and Combine Click-SELEX

For use in the multimodal click-SELEX method, the non-natural nucleotide EdU, which was incorporated into the DNA library, was modified with copper (I) alkine-azide cycloaddition using the CuAAC or “click” reaction as described above with different functionalisations, 3-ethyl-1H-indole (In), 1-methyl-benzene (Bn), 4-ethyl-phenol (Phe) and N-ethyl-guanidine (Gua). The starting library was modified via CuAAC with the four azides individually, pooled subsequently and used for the next SELEX cycle including selection with washing steps, elution of the DNA/protein complex and subsequent PCR. Here the modified DNA can be used as a template for the polymerase. Thus, the modification is removed and alkyne bearing DNA is amplified. This ds DNA is digested to ssDNA by λ-exonuclease (SSD) and the ssDNA is used for the click-modification. For the assignment of the sequences (single azide selection steps) to the respective modification needed for binding, the ssDNA is split into five samples, one remained unmodified, whereas the other four are click modified. This DNA is than individually used for the next two selection cycles.

The Split and combine click-SELEX reactions were performed as described above. Cycles 1-11 were done as multimodal selection and cycle 12 and 13 as single azide selections. Starting from the first cycle 1 to 11, 125 pmol of the respective click competitor was used (500 pmol in total) in cycles 12 and 13 500 pmol of the respective click competitor was added. Starting from cycle 2, DNA was incubated first with 50 μL empty beads as counter selection step. The selection details of the selection cycles are summarised in Table 3 below.

TABLE 3
Overview of CXCL9 multimodal selection

| | | | salmon | | | | | | |
|---|---|---|---|---|---|---|---|---|---|
| | | | sperm | dextran | CXCL9 | | | wash | |
| | DNA | BSA | DNA | sulfate | beads | incubation | | volume | PCR |
| cycle | [pmol] | [mg/mL] | [mg/mL] | [mg/ml] | [pmol] | [min] | wash time | [μL] | cycles |
| 1 | 4 × 125 | 0.2 | 0.1 | | 328 | 30 | 1x short | 100 | 8 |
| | | | | | | | 2x 3 min | | |
| 2 | 31 | 0.2 | 0.1 | | 328 | 30 | 1x short | 100 | 10 |
| | | | | | | | 2x 3 min | | |
| 3 | 18 | 0.6 | 0.1 | | 164 | 25 | 1x short | 150 | 12 |
| | | | | | | | 2x 5 min | | |
| 4 | 18 | 0.6 | 0.25 | | 164 | 20 | 1x short | 200 | 11 |
| | | | | | | | 3x 5 min | | |
| 5 | 10 | 0.6 | 0.25 | | 82 | 15 | 1x short | 250 | 12 |
| | | | | | | | 3x 7.5 min | | |
| 6 | 15 | 1.2 | 0.1 | | 41 | 10 | 1x short | 300 | 15 |
| | | | | | | | 3x 10 min | | |
| 7 | 14 | 1.2 | 0.1 | 0.01 | 6.6 | 5 | 1x short | 300 | 12 |
| | | | | | | | 5x 10 min | | |
| 8 | 9 | 1.2 | 0.1 | 0.1 | 1.3 | 5 | 1x short | 500 | 15 |
| | | | | | | | 15x 5 min | | |
| 9 | | 1.2 | 0.1 | 1 | 0.33 | 5 | 1x short | 400 | 14 |
| | | | | | | | 15x 5 min | | |
| 10 | 11 | 1.2 | 0.1 | 1 | 0.007 | 5 | 1x short | 500 | 14 |
| | | | | | | | 15x 5 min | | |
| 11 | 13 | 1.2 | 0.1 | 1 | 0.007 | 5 | 1x short | 600 | 14 |
| | | | | | | | 15x 5 min | | |
| 12 | 10 | 1.2 | 0.1 | 1 | 0.007 | 5 | 1x short | 600 | 18 |
| E | | | | | | | 15x 5 min | | |
| 12 | 4 | 1.2 | 0.1 | 1 | 0.007 | 5 | 1x short | 600 | 12 |
| In | | | | | | | 15x 5 min | | |
| 12 | 5 | 1.2 | 0.1 | 1 | 0.007 | 5 | 1x short | 600 | 14 |
| Bn | | | | | | | 15x 5 min | | |
| 12 | 5 | 1.2 | 0.1 | 1 | 0.007 | 5 | 1x short | 600 | 12 |
| Phe | | | | | | | 15x 5 min | | |
| 12 | 4 | 1.2 | 0.1 | 1 | 0.007 | 5 | 1x short | 600 | 18 |
| Gua | | | | | | | 15x 5 min | | |
| 13 | 12 | 1.2 | 0.1 | 1 | 0.007 | 5 | 1x short | 600 | 16 |
| E | | | | | | | 15x 5 min | | |
| 13 | 10 | 1.2 | 0.1 | 1 | 0.007 | 5 | 1x short | 600 | 11 |
| In | | | | | | | 15x 5 min | | |
| 13 | 7 | 1.2 | 0.1 | 1 | 0.007 | 5 | 1x short | 600 | 12 |
| Bn | | | | | | | 15x 5 min | | |
| 13 | 12 | 1.2 | 0.1 | 1 | 0.007 | 5 | 1x short | 600 | 11 |
| Phe | | | | | | | 15x 5 min | | |
| 13 | 8 | 1.2 | 0.1 | 1 | 0.007 | 5 | 1x short | 600 | 18 |
| Gua | | | | | | | 15x 5 min | | |

Interaction analysis was performed using flow cytometry as described above of the starting library binding to CXCL9 immobilised on beads and after 11 or 13 selection cycles. For this, 500 nM Cy-5 labelled DNA from the starting library (SL) as well as from selection cycle 11 and selection cycle 13 were incubated with CXCL9 immobilised on magnetic beads. DNA was either unmodified (E) or click modified with indole (In), benzyl (Bn), phenol (Phe) or guanidine (Gua) azide.

The FIG. 1 shows the results of the interaction analysis (n=2-4, mean±SD). As can be taken from FIG. 1, after 11 and 13 selection cycles no binding of unmodified DNA was seen, but enhanced binding of cycle 11 compared to the starting library was seen with indole, benzyl and phenol modifications.

After 13 selection cycles, each of the obtained DNA libraries from selection cycles 0, 4, 7, 9, 11 and 13 was analysed by next-generation sequencing (NGS) as described above. FIG. 2 shows the NGS results of the two analysed sequences SEQ ID NO: 4, in the following denoted G123, and SEQ ID NO: 5, in the following denoted 129. FIGS. 2a) and 2b) show frequency and fold change, respectively, of cycles 9, 11 and 13 of the sequence G123. FIGS. 2c) and 2d) show frequency and fold change of cycles 9, 11 and 13 of sequence 129. As can be taken from FIG. 2, the sequences G123 (SEQ ID NO: 4) and I29 (SEQ ID NO: 5), were found to be enriched.

### Example 2

### Determination of Binding of the Aptamers of SEQ ID NO: 4 and 6 to CXCL9 Using Flow Cytometry

The interaction analysis with CXCL9 was performed using flow cytometry as described above. 500 nM Cy-5 labelled DNA from the starting library (SL) as well as from selection cycle 11 (c11) and the aptamer of SEQ ID NO: 4 and SEQ ID NO: 6 (G123) were incubated with CXCL9 immobilised on magnetic beads. The DNA used was either conventional DNA (DNA, SEQ ID NO: 6), unmodified E-dU (E) or click modified with indole (In), benzyl (Bn), phenol (Phe) or guanidine (Gua) azide. FIG. 3 shows the fluorescence results of the aptamer of SEQ ID NO: 4 denoted G123 compared to the starting library (SL) and selection cycle 11 (c11) (n=2-4, mean±SD). As can be seen in FIG. 3, the aptamer G123 showed binding to CXCL9 when modified with indole, benzyl, phenol and guanidine as well as in form of unmodified, conventional DNA (SEQ ID NO: 6).

### Example 3

### Determination of Affinity of the Aptamers of SEQ ID NO: 4 and 5 to CXCL9 Using ELONA

Affinity determination and the influence of modifications on the binding of the aptamer 129 to CXCL9 was further analysed using enzyme linked oligonucleotide assay (ELONA). Biotin labelled DNA was detected by colorimetric change using streptavidin-horseradish peroxidase (SA-HRP) as described above using 100 nM aptamer.

The influence of 10 different modifications on affinity of aptamer SEQ ID NO: 5 (I29) was determined: indole (In), benzyl (Bn), phenol (Phe), guanidine (Gua), imidazole (Imi), methylpropane (MePro), benzothiophene (Thio), benzofurane (BnFu), naphthalene (Naph) and ethylamine (Am) or azide without modification (E). FIG. 4 shows the results of the binding of 100 nM 129 with the 10 different modifications to CXCL9, where the binding was normalized to the binding with In-dU, (n=2, mean±SD). As can be taken from FIG. 4, binding of the aptamer 129 to CXCL9 requires indole, benzofurane, naphthalene, phenol or benzothiophene modifications.

In parallel, the influence of indole (In) modification and azide without modification (E) on the affinity of aptamer SEQ ID NO: 4 (G123) was determined using ELONA and compared to the affinity of unmodified DNA (DNA). The following table 4 summarises the binding of G123 and I29 and the affinity constants determined with ELONA (n=2, mean±SD). In the table, “+” represents successful binding, while “−” means no detection of binding, “n.d.” denotes not determined.

TABLE 4
Binding and affinity constants of aptamer SEQ ID NO: 4 (G123)
and aptamer SEQ ID NO: 5 (129) with different modifications

| Sequence | Modification | Binding | KD [nM] |
|---|---|---|---|
| G123 | DNA | + | 92 ± 14 |
| | E | + | n.d. |
| | In | + | 39 ± 5 |
| | Bn | + | n.d. |
| | Phe | + | n.d. |
| | Gua | + | n.d. |
| I29 | E | − | |
| | In | + | 12 ± 2 |
| | Phe | + | 177 ± 28 |
| | BnFu | + | 17 ± 4 |
| | Naph | + | 21 ± 4 |
| | Thio | + | n.d. |
| | Gua | − | |
| | Imi | − | |
| | MePro | − | |
| | Am | − | |

These results show that two highly affine aptamers were selected, where the aptamer of SEQ ID NO: 5 (I29) binds if modified with aromatic groups such as indole, while the aptamer of SEQ ID NO: 4 (G123) binds as DNA (SEQ ID NO: 6) but also with different modifications.

### Example 4

### Determination of Specificity of Binding of Aptamers of SEQ ID NO: 4 and 5 to CXCL9 and CXCL11

Binding of aptamers of SEQ ID NO: 4 (G123) and SEQ ID NO: 5 (I29) to different proteins was determined using ELONA. Biotin labelled DNA (conc=2×KD) of unmodified aptamer G123 (DNA, SEQ ID NO: 6), indole-modified aptamer G123 (G123 In) and indole-modified aptamer 129 (I29 In) was detected by colorimetric change using streptavidin-horseradish peroxidase as described above. The pro-inflammatory chemokines CXCL9, CXCL10, CXCL11, and CXCL1 were used, as well as soluble interleukin-2 receptor (sCD25), C-reactive protein (CRP), C—C motif chemokine ligand 17 (CCL17), C—C motif chemokine ligand 22 (CCL22), C—C motif chemokine ligand 3(CCL3) and human serum albumin (HAS).

The following table 5 summarises the binding of unmodified aptamer G123 (DNA, SEQ ID NO: 6), indole-modified aptamer G123 (G123 In) and indole-modified aptamer 129 (I29 In). In the table, “+” represents successful binding, while “−” means no detection of binding.

TABLE 5
Binding of G123 and I29 to different proteins.

| | G123 DNA | G123 In | 129 In |
|---|---|---|---|
| Protein | binding | binding | binding |
| CXCL9 | + | + | + |
| CXCL10 | − | − | − |
| CXCL11 | + | + | + |
| CXCL1 | − | − | − |
| sCD25 | − | − | − |
| CRP | − | − | − |
| CCL17 | − | − | − |
| CCL22 | − | − | − |
| CCL3 | − | − | − |
| HSA | − | − | − |

As can be seen from table 5, next to pro-inflammatory chemokine CXCL9 also CXCL11 is recognized by the aptamer G123, either unmodified or indole-modified, and indole-modified aptamer 129. The binding results shows that both aptamer of SEQ ID NO: 5 (I29) and SEQ ID NO: 4 (G123) are highly selective aptamers, showing affinity to CXCL9, but no binding to CXCL10, although all three chemokines bind to the same receptor (CXCR3).

The following table 6 summarises the affinity constants of the binding of unmodified aptamer G123 (DNA, SEQ ID NO: 6), indole-modified aptamer G123 (G123 In) and indole-modified aptamer 129 (I29 In) to pro-inflammatory chemokines CXCL9 and CXCL11 determined with ELONA (n=2, mean±SD).

TABLE 6
Affinity constants of G123 to CXCL11 and CXCL9

| Sequence | KD CXCL11 [nM] | KD CXCL9 [nM] |
|---|---|---|
| G123 DNA | 249 ± 44 | 92 ± 14 |
| G123 In | 73 ± 15 | 39 ± 5 |
| I29 In | 17 ± 2 | 12 ± 2 |

The affinity constants show that two highly affine aptamers for pro-inflammatory chemokines CXCL9 and CXCL11 could be identified.

### Example 5

### Analysis of Binding Motif

### 5.1 Analysis of Importance of Thymidines for Binding to CXCL9

The influence of substitutions of the nucleosides at positions 28, 44, 47, 49 and 50 of the aptamer of SEQ ID NO: 5 was analysed in view of their importance for binding of the aptamers to CXCL9. For this, in the aptamer sequence of SEQ ID NO: 5 (I29) the Ed-U modifications were substituted with conventional Ts and binding to CXCL9 was determined using enzyme linked oligonucleotide assay (ELONA) where Biotin labelled DNA (100 nM) of 129 variants was detected by colorimetric change using streptavidin-horseradish peroxidase (SA-HRP) at 405 nm.

FIG. 5A shows the effect of substitution of the Ed-U modifications with conventional thymidines (Ts) on binding to CXCL9 (n=2, mean±SD). As can be seen in FIG. 5A, except for position 28, all modifications are important for binding to CXCL9. Further, it can be seen that the indole modification provides the highest stability. FIG. 5B shows the frequency of the 129 motif (CGACNXACXGXXCAG (SEQ ID NO: 16); X=Ed-U, N=any base) enrichment during CXCL9 SELEX of example 1.

### 5.2 G-Quadruplex Characterisation

For further characterisation, a G-quadruplex characterisation of the sequence of the aptamer of SEQ ID NO: 4 (G123) was performed using flow cytometer binding assay. 100 nM Cy-5 labelled G123 was incubated with CXCL9 magnetic beads. DNA was either conventional DNA (DNA, SEQ ID NO: 6) or click modified with indole (In), benzyl (Bn), phenol (Phe) or guanidine (Gua) azide. Incubation was done in different buffer conditions, i.e. with kalium kation (K⁺) in the binding buffer, without kalium kation (w/o K⁺), or K⁺ and NH₄OAc (w/o K⁺& NH₄OAc).

FIG. 6A shows the results of the flow cytometer binding assay (n=2-5, mean±SD, binding normalized to binding in SELEX conditions (w/K⁺)). As can be seen in FIG. 6A, G123 DNA cannot bind without K⁺ in the binding buffer, whereas click modified DNA can still bind to CXCL9, even without K⁺ and NH₄OAc. FIG. 6B shows the circular dichroism analysis of G123 DNA in water and in PBS. FIG. 6B confirms the G-rich quadruplex motif. This shows that the G-rich motif of SEQ ID NO: 1 is important for binding to the pro-inflammatory chemokine CXCL9.

### Example 6

### Determination of Binding to CXCL9 Epitope Using Competition Assays

The binding of indole-modified aptamer of SEQ ID NO: 5 (I29 In), unmodified aptamer of SEQ ID NO: 6 (G123 DNA), and indole-modified aptamer of SEQ ID NO: 4 (G123 In) to pro-inflammatory chemokine CXCL9 was determined in competition assay. 100 nM Cy-5 labelled DNA was incubated simultaneous with 1 μM unlabelled DNA and thus competed for binding. After incubation the remaining Cy5 labelled DNA on CXCL9-magnetic beads was analysed with flow cytometry as described above. As control, Cy5-labeled DNA without competitor was analysed and was used to normalize data.

FIG. 7 shows the results of the competition assay of sequence 129 In, G123 DNA and G123 In. Binding of Cy5 labelled scrambled sequence is used as non-binding control. (n=2, mean±SD). As can be taken from FIG. 7A, competition of G123 DNA can be mainly seen for all 3 sequences (G123, G123 (In) and I29 (In) but also to some parts of the scrambled variants. FIG. 7B shows the competition results for unmodified aptamer of SEQ ID NO: 6 (G123 DNA). FIG. 7C shows the competition results for modified aptamer of SEQ ID NO: 5 (I29 In). As can be taken from FIG. 7C, competition of of 129 (In) can solely be seen for 129 (In), G123 (In) and G123.

This shows that the aptamers probably bind to the same epitope on CXCL9. In a diagnostic test for detecting the chemokine CXCL9, the aptamers may be used in a sandwich-complex with antibodies to CXCL9, which bind to different epitopes. Sandwich-complexes containing both aptamers G123 and I29 seem less useful as both aptamers compete for the binding epitope.

### Example 7

### Determination of Binding Conditions to CXCL9 in Buffer and Urine

### 7.1 Binding of Aptamers to CXCL9 in Buffers of Different pH Values

Binding of aptamers to CXCL9 in buffer of different pH values was characterised using flow cytometer binding assay. 100 nM Cy-5 labelled aptamers were incubated with CXCL9 magnetic beads in buffers of pH values of 5.2, 6.5, 7.3, 8.3 and 9.0. The aptamer sequences tested were either conventional aptamer of SEQ ID NO: 6 (G123 DNA), or the aptamer of SEQ ID NO: 4 click-modified with indole azide (G123), or the aptamer of SEQ ID NO: 5 click-modified with indole azide (I29).

FIG. 8A shows the binding to CXCL9 depending on buffer conditions (n=3-4, SD±mean, binding normalized to binding in SELEX conditions (pH 6.5)). As can be seen in FIG. 8A, binding of indole-modified G123 was more stable over a broad pH range, compared to unmodified G123 and indole-modified 129.

### 9 7.2 Binding of Aptamers to CXCL9 in Human Urine

The binding of the conventional aptamer of SEQ ID NO: 6 (G123 DNA), the aptamer of SEQ ID NO: 4 click-modified with indole azide (G123 In-dU), and the aptamer of SEQ ID NO: 5 click-modified with indole azide (I29 In-dU) to CXCL9 was further analysed in SELEX buffer of pH 6.5 and in untreated human urine from a healthy donor using ELONA binding assay of biotin labelled aptamers. FIG. 8B shows the binding to CXCL9 in SELEX buffer of pH 6.5 and in human urine (n=2, mean±SD, binding normalized to binding in SELEX buffer). As can be seen in FIG. 8B, binding of all sequences was reduced in urine, but still was distinguishable from control sequences.

This shows that the aptamers are usable in human urine for diagnostic tests to detect the chemokines CXCL9 and CXCL11 in urine of kidney allograft patients.

### Example 8

### Determination of the Binding of Aptamers to Endogenous CXCL9

The binding of the aptamers was further analysed in cell supernatant from human peripheral blood mononuclear cells. Cell supernatant was generated from isolated PBMC's (peripheral blood mononuclear cells) from blood donors that were differentiated for 7 days to macrophages. Macrophages were stimulated with Interferon-gamma (10 ng/ml) and LPS (lipopolysaccharide, 1 μg/ml) for 24-48 h in RPMI 1640 Medium+5% humane AB Serum+1% Penicillin/Streptomycin+25 ng/ml m-CSF. Cell supernatant was collected and directly stored at −80° C.

The assay was performed as sandwich assay. CXCL9 specific antibody was immobilized on magnetic beads. This antibody-protein-beads complex were incubated with recombinant CXCL9 in control cell supernatant (rec) and native CXCL9 (native) or control supernatant without CXCL9 (ctrl) in cell supernatant and analysed with flow cytometry. The CXCL9 concentration used in this assay was 0.9 nM (11 ng/ml) which is lower than the CXCL9 concentration in urine of patients with renal rejection that was reported to be 14 nM (178 ng/ml). After washing, 150 nM Cy-5 labeled DNA was used to detect the resulting complex as depicted in FIG. 10.

The binding of 150 nM of the respective Cy-5 labeled aptamers of SEQ ID NO: 5 click-modified with indole azide (I29 In-dU), the unmodified, conventional aptamer of SEQ ID NO: 6 (G132 DNA) and the aptamer of SEQ ID NO: 4 click-modified with indole azide (G123 In-dU) was tested.

FIG. 9 shows the results of the binding of CXCL9 aptamers to endogenous CXCL9 for native CXCL9 in cell supernatant (native), recombinant CXCL9 in control cell supernatant (rec) and control supernatant without CXCL9 (ctrl) with Cy5-labelled 129 In-dU in FIG. 9A, G132 DNA in FIG. 9B and G123 In-dU in FIG. 9C. Binding was normalized to recombinant protein in control supernatant (n=2, mean±SD). As can be taken from FIG. 9, all aptamers can bind the endogenous CXCL9, even if binding was reduced compared to recombinant CXCL9.

### Example 9

Design and Testing of an Antibody-Aptamer-Hybrid Lateral Flow Assay for the Detection of CXCL9 in Antibody-Mediated Rejection (AMR) after Kidney Transplantation

Based on the binding of the aptamers to endogenous CXCL9 an aptamer-antibody-hybrid lateral flow assay (hybrid-LFA) for detection of CXCL9 in urine was developed. The CXCL9-specific hybrid-LFA was developed based upon a specific rat antibody immobilized on a nitrocellulose-membrane and the coupling of the CXCL9-binding aptamer G123 to gold nanoparticles.

The FIG. 11 shows a schematic view on the aptamer-antibody-hybrid lateral flow assay (hybrid-LFA) designed as a sandwich assay with a capture antibody membrane-immobilized in the test zone and a detecting aptamer coupled to AuNPs. As shown in FIG. 11, a short oligonucleotide sequence complementary to the aptamer was immobilized in the control zone. CXCL9 in a sample applied binds to the AuNP-labeled detection aptamer (AuNP-G123) and this complex was fixed on the test zone by binding to the capture antibody. In case of sufficient CXCL9 is bound, the marker line turns red and is visible. Without target CXCL9, no marker line appears. When reaching the control zone, the oligonucleotide/AuNP-G123 complex forms a control line indicating that the test is functional.

### 9.1 Materials and Further Components of the Lateral Flow Assay

Nitrocellulose membrane Unisart® CN95 was kindly provided by Sartorius Stedim Biotech (Goettingen, Germany). Gold nanoparticles (AuNP) were kindly provided by Fassisi GmbH (Goettingen, Germany). Commercial CXCL9 (Cat #TP720369) was acquired from OriGene (MD, USA). Trehalose was purchased from Fluka BioChemika (Steinheim, Germany). Sucrose and bovine serum albumin (BSA) were obtained from Sigma-Aldrich GmbH (Munich, Germany). Tween20 was purchased from PanReac AppliChem (Darmstadt, Germany). Phosphate buffered saline (PBS, pH 7.4) was prepared. Blocking buffer was based upon PBS supplemented with 10% fetal calf serum (FCS) (Sigma-Aldrich Chemie GmbH, Taufkirchen, Germany). Binding buffer (BB, pH 6.5) was PBS-based with its content adjusted to urine milieu, supplemented with 0.25% BSA (Sigma-Aldrich GmbH), 0.1 mg·ml⁻¹ salmon sperm DNA (Thermo Fisher, MA, USA).

### 9.2 Antibody

An antibody exhibiting a high affinity for CXCL9 was generated by Helmholtz Zentrum Muenchen (HZI, Germany) based upon standardized immunization of rats with commercial CXCL9 (OriGene, MD, America) and using the mouse myeloma cell line P3X63-Ag8.653. 9.3 Preparation of gold nanoparticle-aptamer conjugates (AuNP-G123) The unmodified aptamer of SEQ ID NO: 6 (G123) was modified with a disulfide linker at the 5′-terminus and extended with a hexaethylene glycol spacer (C6-SS-HEG-5′-G123-3). The preparation of the gold nanoparticle-aptamer conjugates (AuNP-G123) was performed according to an adjusted protocol as described of Phung et al. (Phung et al. “Development of an Aptamer-Based Lateral Flow Assay for the Detection of C-Reactive Protein Using Microarray Technology as a Prescreening Platform” ACS combinatorial science 2020; 22: 617-629).

After conjugation of aptamers the AuNP surface exhibited an increased diameter of nanoparticles without signs of agglomeration. The altered size of nanoparticles was verified using dynamic light scattering (DLS, Particle Analyzer Litesizer™ 500, Anton Paar, Graz, Austria) and UV-Vis (Epoch Mikroplatten-Spektralphotometer, BioTek Instruments, Winooski, USA). The AuNP-G123 was compared to unmodified AuNPs exhibiting enlargement of particles due to the density distribution of particle diameter and the right shift of the wavelength. Functionality of AuNP-G123 was verified via lateral flow assay and non-specific binding partners were applied for a comparison (human serum albumin (HSA), sCD25, CRP were used as negative controls).

### 9.4 Preparation of Test Strips for the Lateral Flow Assay

The hybrid-lateral flow assay was composed of a sample pad (Åhlstrom-Munksjö, Helsinki, Finland) and an absorbent pad (Åhlstrom-Munksjö, Helsinki, Finland, grade 222) glued to a backing card (GE-Whatman, 7.5 cm) with 2 mm overlap to the centered nitrocellulose membrane. Sample pads were incubated for 2 h with 100 mM Tris (pH 8.0) supplemented with 1% BSA, 1% sucrose and 0.05% Tween 20 and dried at ambient temperature overnight before they were applied on the backing cards. Solutions for test (A, 1 mg·ml⁻¹) and control zone were generally automatically applied onto the nitrocellulose membrane (Fassisi GmbH, Goettingen, Germany, 1 μL·cm⁻¹), in some cases by hand (0.2 μL). Beforehand, streptavidin (20 μM in PBS, Roth, Germany) and the same volume of biotinylated oligonucleotide solution (20 μM in PBS) was mixed and incubated for 2 h at 500 rpm to prepare the solution for the control zone. Test und control zones were dried for 2 h at 50° C., cut into strips of 4 mm and placed in an airtight bag under exclusion of light.

### 9.5 Sample Preparation and Test Runs

100 μL of each sample (technical sample, spiked sample or patient sample) were placed at ambient temperature in a well of a 96-well plate. 1 μL AuNP-G123 was applied onto the conjugate pad. Concentrations of purified CXCL9 were diluted in binding buffer (technical sample) or in binding buffer combined with pooled patient negative urine samples (spiked samples) derived of four kidney transplant recipients with non-rejection proven by biopsy. All patient samples were diluted in binding buffer (ratio 1:1). Lateral flow assay strips were placed in a sample well and scanned (Epson V370) after 15, 30 and 45 minutes before quantitative analysis by eye as well as by ImageJ-estimation of scans (ImageJ 1.46r, NIH, USA).

9.6 LFA Evaluation with Technical and Spiked Samples

To exclude non-specific LFA performance, a scrambled oligonucleotide exhibiting the same composition of bases as G123 but arranged in a different primary structure as well as unmodified AuNPs were used.

For determination of the limit of detection (LOD) of the LFA according to red color signals in the test zone, the system was exposed to technical samples using a serial dilution of purified CXCL9 in binding buffer (0-300 pg·ml⁻¹) and different running times (15 to 45 minutes). An LOD of 10 pg·ml⁻¹ was determined with a running time of 45 minutes. Spiked samples were prepared by using a 1:1 ratio of pooled urine samples of patients with unsuspicious finding proven by biopsy and binding buffer since pretests showed best results. As compared to the test performance with technical samples, here an LOD of 60 pg·ml⁻¹ CXCL9 was determined.

9.7 Detection of CXCL9 in Patients with Antibody-Mediated Rejection (AMR)

Detection of CXCL9 was performed in 48 human urine samples from kidney transplant recipients, transplanted between 1986 and 2017, with antibody-mediated rejection (AMR) (23 patients) and non-rejection (25).

As illustrated in FIG. 12, the scans after a running time of 15 minutes showed distinct red lines in the according zone for patient samples. Lateral flow assays with a signal intensity of the test zone lower than 200 AU according to ImageJ did not reveal a visible line in the test zone. Therefore, 200 AU was defined as threshold (shown as horizontal dashed line in FIG. 12). As can be taken from the results shown in FIG. 12, 17 of 23 AMR samples and 6 of 25 urine samples of patients with non-rejection showed a positive signal. The lateral flow assay specificity and sensitivity was therefore determined to be 71% for each parameter.

Hybrid-lateral flow assay performance was assessed according to receiver operating characteristic (ROC) analysis. ROC analyses were performed to display the area under the curve (AUC) as well as the sensitivity and specificity of the LFA as compared to the estimated glomerular filtration rate at the day of biopsy. As illustrated in FIG. 13, the ROC analyses revealed that the AUC of the lateral flow assay was higher as compared to estimated glomerular filtration rate on the day of biopsy (lateral flow assay: AUC=0.799 vs. eGFR: AUC=0.428). The positive predictive value of the new test was 0.652 and the negative predictive value was 0.708.

In summary, the hybrid-lateral flow assay provided a sensitivity and specificity of 71% and an AUC of 0.79 for CXCL9. This provides an improvement in early diagnosis-making in AMR after kidney transplantation, especially in kidney transplant recipients with undetermined status of donor-specific HLA-antibodies.

1. An aptamer comprising a nucleotide sequence 5′-N₁N₂GN₃CCN₁AN₄N₅N₁N₆N₇N₈N₁-3′ (SEQ ID NO: 1) or pharmaceutically acceptable salts thereof, wherein:
N₁ represents T or 5-ethynyl-2′-deoxyuridine (EdU) which is modified with a side chain selected from the group consisting of an indole, benzofurane, naphthalene, phenol, benzothiophene, guanidine and benzyl,
N₂ represents a sequence of 9 or 11 contiguous nucleotides comprising at least 5 guanidine nucleotides,
N₃ represents A or C,
N₄ represents A or a deletion,
N₅ represents C or G,
N₆ represents C or a deletion,
N₇ represents C or G,
N₈ represents G or N₁.

2. The aptamer according to claim 1, wherein the aptamer comprises a nucleotide sequence 5′-CACGACGCAAGGGACCACAGGGAGGGAGGGN₁GGGC AAAGGGCCCN₁AAGN₁CCGN₁AACAAAAACACAGCACGACACCGCAGAGGCA-3′ (SEQ ID NO: 4) or pharmaceutically acceptable salts thereof, wherein N₁ represents T or EdU which is modified with a side chain selected from the group consisting of an indole, phenol, guanidine and benzyl.

3. The aptamer according to according to claim 1, wherein the aptamer comprises a nucleotide sequence 5′-CACGACGCAAGGGACCACAGGGAGGGA GGGTGGGCAAAGGGCCCTAAGTCCGTAACAAAAACACAGCACGACACCGCAGAGG CA-3′ (SEQ ID NO: 6) or a nucleotide sequence SEQ ID NO: 4, wherein N₁ represents EdU which is modified with an indole, or pharmaceutically acceptable salts thereof.

4. The aptamer according to claim 1, wherein the aptamer comprises a nucleotide sequence 5′-CACGACGCAAGGGACCACAGGAGAGACN₁CACGGG CGGGCGACCN′₁ACN′₁GN′₁N′₁CAGCCCAGACCGACAGCACGACACCGCAGAGGCA-3′ (SEQ ID NO: 5) or pharmaceutically acceptable salts thereof, wherein N₁ represents T or EdU which is modified with an aromatic group selected from the group consisting of an indole, benzofurane, naphthalene, phenol and benzothiophene and N′₁ represents EdU which is modified with an aromatic group selected from the group consisting of an indole, benzofurane, naphthalene, phenol and benzothiophene.

5. The aptamer according to claim 4, wherein the aromatic group is selected from the group consisting of an indole, benzofurane and naphthalene.

6. An aptamer comprising a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and pharmaceutically acceptable salts thereof, wherein the aptamer is used as a medicament or a diagnostic reagent.

7. The aptamer according to claim 6, wherein the aptamer is used in the detection or diagnosing of a rejection of a renal allograft.

8. The aptamer according to claim 6, wherein the aptamer is in a complex with an antibody against chemokines CXCL9 or CXCL11.

9. A diagnostic composition comprising the aptamer of claim 6 as the diagnostic reagent, wherein the aptamer comprises a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and pharmaceutically acceptable salts thereof.

10. A diagnostic test comprising the aptamer of claim 6 as the diagnostic reagent.

11. (canceled)

12. A pharmaceutical composition comprising the aptamer of claim 6 as an active ingredient, wherein the aptamer comprises a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and pharmaceutically acceptable salts thereof.

13. A method of manufacture of a medicament or a diagnostic reagent, comprising providing the aptamer of claim 6.

14. An in vitro method of detecting or diagnosing a rejection of a renal allograft, the method comprising the steps of:
1) selecting an aptamer comprising a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and pharmaceutically acceptable salts thereof
2) detecting the binding of the aptamer to CXCL9 or CXCL11 in a sample obtained from a subject.

15. The method of claim 14, wherein the sample is selected from urine or serum of a renal allograft patient.

16. The diagnostic test of claim 10, wherein the test is used for the detection or diagnosing of a rejection of a renal allograft.

17. The method of manufacture of claim 13, wherein the aptamer is used in the detection or diagnosing of a rejection of a renal allograft.

18. A method of detecting or diagnosing a rejection of a renal allograft comprising providing the diagnostic composition of claim 9 and using the diagnostic composition to detect or diagnose the rejection of the renal allograft.

19. The aptamer of claim 3, wherein the aptamer consists of a nucleotide sequence of SEQ ID NO: 6 or a nucleotide sequence SEQ ID NO: 4, wherein N1 represents EdU which is modified with an indole, or pharmaceutically acceptable salts thereof.

20. The aptamer of claim 6, wherein the aptamer consists of a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 and pharmaceutically acceptable salts thereof, wherein the aptamer is used as a medicament or a diagnostic reagent.
